(19) United States
(12) Patent Application Publication
(10) Pub. No.: US 2016/0237052 A1
(43) Pub. Date: Aug. 18, 2016

(54) Histamine-3 Receptor Antagonists

(71) Applicants: Dennis Michael Godek, Glastonbury, CT (US); Harry Ralph Howard, Bristol, CT (US)

(72) Inventors: Dennis Michael Godek, Glastonbury, CT (US); Harry Ralph Howard, Bristol, CT (US)

(73) Assignee: MediSynergics, LLC, Newington, CT (US)

(21) Appl. No.: 14/624,821

(22) Filed: Feb. 18, 2015

(51) Int. Cl. C07D 307/87

(52) U.S. Cl. CPC C07D 307/87

(57) ABSTRACT

The invention is directed to a compound of formula I, as defined herein, or a pharmaceutically acceptable salt thereof; a pharmaceutical composition containing a compound of formula I; and a method of treatment of a disorder or condition selected from the list consisting of acute myocardial infarction; memory processes disorders; dementia; cognition disorders such as Alzheimer's disease; attention deficit disorder (ADD); attention-deficit hyperactivity disorder (ADHD); cancers such as cutaneous carcinoma, medullary thyroid carcinoma and melanoma; Meniere's disease; gastrointestinal disorders; inflammation; migraine; motion sickness; obesity; pain; septic shock; respiratory disorders (including allergic rhinitis, nasal congestion and allergic congestion) in a mammal, including a human, that may be effected by administering to said mammal a pharmacologically effective amount of a compound of formula I, or a pharmaceutically acceptable salt thereof, and a pharmaceutically acceptable carrier.

This application claims the benefit of U.S. Provisional Application Ser. No. 61/941,139 filed on Feb. 18, 2014.

### BACKGROUND OF THE INVENTION

This invention is directed to compounds of formula (I) described herein, to a pharmaceutical composition comprising such compounds, and to methods of treatment of disorders or conditions that may be treated by antagonizing histamine-3 (H3) receptors using such compounds. The histamine-3 (H3) receptor antagonists of the invention are useful for treating anxiety disorders, including for example, generalized anxiety disorder (GAD), panic disorder, posttraumatic stress disorder (PTSD), and social anxiety disorder; mood adjustment disorders, including depressed mood, mixed anxiety and depressed mood, disturbance of conduct, and mixed disturbance of conduct and depressed mood; age-associated learning and mental disorders, including Alzheimer's disease; attention adjustment disorders such as attention deficit disorders, or other cognitive disorders due to general medical conditions; psychotic disorders, including schizoaffective disorders and schizophrenia; sleep disorders, including narcolepsy and enuresis; obesity; dizziness, epilepsy, and motion sickness. The H3 receptor antagonists of the invention are also useful for treating, for example, allergy, allergy-induced airway (e.g., upper airway) responses, congestion (e.g., nasal congestion), hypotension, cardiovascular disease, diseases of the gastrointestinal (GI) tract, hyper- and hypo-motility and acidic secretion of the GI tract, sleeping disorders (e.g., hypersomnia, somnolence and narcolepsy), disturbances of the central nervous system (CNS), including attention deficit hyperactivity disorder (ADHD), hypo- and hyperactivity of the CNS system (e.g., agitation and depression), and other CNS disorders (such as schizophrenia and migraine).

Histamine is a well-known mediator in hypersensitive reactions (e.g., allergies, hay fever and asthma) that are commonly treated with antagonists of histamine (i.e., antihistamines). It has been established that histamine receptors exist in at least two distinct subtypes, referred to as H1 and H2. A third histamine receptor (H3 receptor) is believed to play a role in neurotransmission in the CNS, where the H3 receptor is thought to be disposed presynaptically on histaminergic nerve endings (Arrang et al, Nature, 302:832-837 (1983)). The existence of the H3 receptor has been confirmed by the development of selective H3 receptor agonists and antagonists (Arrang et al, Nature, 327:117-123 (1987) and has subsequently been shown to regulate the release of the neurotransmitters in both the CNS and peripheral organs, particularly the lungs, cardiovascular system and the GI tract.

A number of diseases or conditions may be treated with histamine-3 receptor ligands wherein the H3 ligand may be an antagonist, agonist or partial agonist; for example, see (Imamura, et al, Circ. Res. 1996, 78:475-481), (Imamura, et al, Circ. Res., 1996, 78:863-869), (Lin, et al, Brain Res., 1990, 523:325-330), (Monti et al, Neuropsychopharmacology, 1996, 15:31-35), (Sakai, et al, Life Sci., 1991, 48:2397-2404), (Mazurkiewiez-Kwilecki and Nsonwah, Can. J. Physiol. Pharmacol., 1989, 67:75-78), (Panula, et al, Neuroscience, 1998, 44:465-482), (Wada, et al, Trends in Neuroscience, 1991, 14:415), (Monti, et al, Eur. J. Pharmacol., 1991, 205:283), (Haas, et al, Behay. Brain Res., 1995, 66:41-44), (de Almeida and Izquierdo, Arch. Int. Pharmacodyn., 1986, 283:193-198), (Kamei, et al, Psychopharmacology, 1990, 102:312-318), (Kamei and Sakata, Japan. J. Pharmacol., 1991, 57:437-482), (Schwartz, et al, Psychopharmacology: The Fourth Generation of Progress, Bloom and Kupfer, (eds.) Raven Press, New York, N.Y., 1995, 3-97), (Shaywitz, et al, Psychopharmacology, 1984, 82:73-77), (Dumery and Blozovski, Exp. Brain Res., 1987, 67:61-69), (Tedford, et al, J. Pharmacol. Exp. Ther., 1995, 275:598-604), (Tedford, et al, Soc. Neurosci. Abstr., 1996, 22:22), (Yokoyama, et al, Eur. J. Pharmacol. 1993, 234:129), (Yokoyama and linuma, CNS Drugs, 1996, 5:321), (Onodera, et al, Prog. Neurobiol., 1994, 42:685), (Leurs and Timmerman, Prog. Drug Res., 1992, 39:127), (The Histamine H3 Receptor, Leurs and Timmerman (Eds.), Elsevier Science, Amsterdam, The Netherlands. 1998), (Leurs, et al, Trends in Pharm. Sci, 1998, 19:177-183), (Phillips, et al, Ann. Rep. Medicinal Chem., 1998, 33:31-40), (Matsubara, et al, Eur. J. Pharmacol., 1992, 224:145), (Rouleau, et al, J. Pharmacol. Exp. Ther., 1997, 281:1085), (Adam Szelag, “Role of histamine H3-receptors in the proliferation of neoplastic cells in vitro”, Med. Sci. Monit., 4(5):747-755, 1998), (Fitzsimons, et al, “Histamine receptors signaling in epidermal tumor cells with H-ras gene alterations”, Inflammation Res., 1998, 47(Suppl. 1):550-51), (Leurs, et al, “The medicinal chemistry and therapeutic potentials of ligands of the histamine H3 receptor”, Progress in Drug Research, 1995, 45:107-165), (R. Levi and N.C.E. Smith, “Histamine H3-receptors: A New Frontier in Myocardial Ischemia”, J. Pharm. Exp. Ther., 2000, 292:825-830), (E. Hatta, et al, “Activation of Histamine H3 receptors inhibits carrier-mediated norepinephrine release in a human model of protracted myocardial ischemia”, J. Pharm. Exp. Ther., 1997, 283:494-500), (H. Yokoyama and K. Iinuma, “Histamine and Seizures: Implications for the Treatment of epilepsy”, CNS Drugs, 1995, 5(5):321-330), (K. Hurukami, H. Yokoyama, K. Onodera, K. Iinuma and T. Watanabe, “AQ-0145, A newly developed histamine H3 antagonist, decreased seizure susceptibility of electrically induced convulsions in mice”, Meth. Find. Exp. Clin. Pharmacol., 1995, 17(C):70-73), (Delaunois, et al, “Modulation of acetylcholine, capsaicin and substance P effects by histamine H3 receptors in isolated perfused rabbit lungs”, Eur. J. Pharmacology, 1995, 277(2-3):243-250) and (Dimitriadou, et al, “Functional relationship between mast cells and C-sensitive nerve fibers evidenced by histamine H3-receptor modulation in rat lung and spleen”, Clinical Science, 1994, 87(2): 151-163).

Recently, Brioni et al (Abbott Laboratories) have described the discovery and potential use of a number of new compounds, including ABT-288, for the treatment of cognitive disorders and Alzheimer's disease (Brioni J, Esbenshade T, et al, J. Pharmacology Expt Ther., 2011, 336:38-46; Esbenshade T, Browman K, et al, Brit. J. Pharmacology, 2008, 154:1166-1181). The same authors have also published a review article on the potential for use of H3 antagonists in treating obesity (Esbenshade T, et al, Molecular Interventions, 2006, 6(2):77-88). The use of H3 receptor agonists in the treatment of obesity and diabetes mellitus is described by Yoshimoto R, et al (PNAS, 2006, Sept. 12, 103(37):13866-13871), and a histamine H3-selective agonist, proxyfan, significantly increased plasma insulin levels via a glucose-independent mechanism (Henry M B, et al, Endocrinology, 2011, 152(3):828-835). Cowart, et al (Eur. J. Pharmacology, 2012, 684:87-94) describe the efficacy of the selective histamine H3 antagonist A-960656 in animal models of osteoarthritis and neuropathic pain. Two informative reviews of novel H3 antagonists have been published describing discovery programs to identify novel CNS therapeutics (see Labeeuw O, et al, Bioorganic & Medicinal Chemistry Letters, 2013, 23:2548-2554; Wager T T, et al, Journal of Medicinal Chemistry, 2011, 54:7602-7620).

Such diseases or conditions include cardiovascular disorders such as acute myocardial infarction, memory processes, dementia and cognition disorders such as Alzheimer's disease and attention-deficit hyperactivity disorder; neurological disorders such as Parkinson's disease, schizophrenia, depression, epilepsy, and seizures or convulsions; cancers such as cutaneous carcinoma, medullary thyroid carcinoma and melanoma; respiratory disorders such as asthma; sleep disorders such as narcolepsy; vestibular dysfunction such as Meniere's disease; gastrointestinal disorders, inflammation, migraine, motion sickness, obesity, pain and septic shock. The histamine H3 receptor as a target for drug discovery has recently been extensively reviewed by Michael Berlin, et al (Journal of Medicinal Chemistry, 2011, 54:26-53).

H3 antagonists have been previously described in, for example, WO-03/050099, WO-02/0769252 and WO-02/012224. The histamine H3 receptor (H3R) regulates the release of histamine and other neurotransmitters, including serotonin and acetylcholine. H3R is relatively neuron specific and inhibits the release of certain monoamines such as histamine. Selective antagonism of H3R raises brain histamine levels and inhibits such activities as food consumption while minimizing non-specific peripheral consequences. Antagonists of the receptor increase synthesis and release of cerebral histamine and other monoamines. By this mechanism, they induce a prolonged wakefulness, improved cognitive function, reduction in food intake and normalization of vestibular reflexes. Accordingly, the receptor is an important target for new therapeutics in Alzheimer disease, mood and attention adjustments, including attention deficit hyperactivity disorder (ADHD), cognitive deficiencies, obesity, dizziness, schizophrenia, epilepsy, sleeping disorders, narcolepsy and motions sickness, and various forms of anxiety.

A significant number of histamine H3 receptor antagonists resemble histamine in that they possess an imidazole ring that may be substituted, as described, for example, in WO-96/38142. Non-imidazole neuroactive compounds such as beta histamines (Arrang J-M, et al, Eur. J. Pharm., 1985, 111:72-84) demonstrate some histamine H3 receptor activity but with poor potency. EP-978512 and EP-982300-A2 disclose non-imidazole alkylamines as histamine H3 receptor antagonists. WO-02/12224 (Ortho-McNeil Pharm.) describes non-imidazole bicyclic derivatives as histamine H3 receptor ligands. Other receptor antagonists have been described in WO-02/32893 and WO-02/06233. A number of novel histamine H3 antagonists from Pfizer Inc. have been disclosed in the patent literature with potential utility in the treatment of cognitive disorders, e.g., depression, mood disorders, anxiety, attention deficit disorder (ADD), attention deficit hyperactivity disorder (ADHD), as well as respiratory disorders (e.g., respiratory diseases, allergic rhinitis, nasal and allergic congestion. These include derivatives of biphenylylmethylamines (WO-2005/105744; US-2005/0245503), diazabicyclic diamines (WO-2006/000914; US-2005/0282811), 1,3-substituted-cycloamino derivatives (WO-2006/011042; US-2006/0014733), 3-biphenylyI-1,2,4-oxadiazoles (WO-2006/011043, US-2006/0019998), azabicyclic ethers (WO-2007/138431), spirochromanes (WO-2007/088462), 1-pyrrolidine-indanes (WO-2007/099423) and spirocyclic amines (WO-2007/063385) as selective antagonists of the Histamine H3 receptor.

The present invention is directed to histamine-3 (H3) receptor antagonists useful for treating one or more of the conditions listed in the preceding paragraphs. The compounds of this invention are selective for the H3 receptor vs. other neurologically relevant receptors; in particular, the compounds of this invention selectively distinguish H3R from the other receptor subtypes, e.g., H1R. In view of the increased level of interest in histamine H3 receptor agonists, inverse agonists and antagonists disclosed in the art, the discovery of novel compounds that interact with the histamine H3 receptor would be a highly desirable contribution to the art. For example, in light of Brioni et al (2011) a new compound which demonstrates high affinity as a histamine H3 receptor antagonist as well as efficacious SERT inhibition could lead to further improvements in cognitive performance for patients with Alzheimer's disease and other dementia. The present invention provides such a contribution to the art, being based on the finding that a novel class of diamines, (I), has a high and specific affinity for the histamine H3 receptor.

Previous publications, including one by J. Hytell, in Progress in Neuro-Psychopharmacology and Biological Psychiatry, 1982, 6(3):277-295 report that the antidepressant drug citalopram displays “ . . . no antagonistic activity toward DA, NA, 5-HT, histamine, GABA or morphine receptors”.

A recent publication (Banala A K, Zhang P, Plenge P, et al, Journal of Medicinal Chemistry, 2013, 56:9709-9724), describes the preparation of citalopram analogs in which the nitrile (i.e., —CN) group has been modified to study the effects of structural changes of this compound on affinity for the serotonin S1 (i.e., 5-HT₁) and S2 (i.e., 5-HT₂) binding sites as well as for the norepinephrine transporter (NET); histaminergic activity is not disclosed nor discussed.

Thus, the starting point for new chemical substances disclosed within this application, the citalopram molecule, does not exhibit any appreciable affinity for the histamine H3 receptor and structural changes to incorporate H3R affinity would not be obvious to one skilled in the art.

### SUMMARY OF THE INVENTION

This invention is directed to a method of use of a compound of the formula (I):

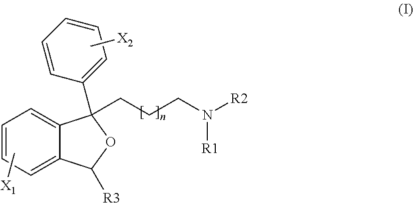

or a pharmaceutically acceptable salt thereof, wherein:

X₁ is a group of the formula (IV):

—(CH₂)ₘ—NR5R6 (IV);

X₂ is H, Cl or F;

R1 and R2 are independently hydrogen or methyl;

R3 is hydrogen or methyl;

m is one;

n is zero, one or two;

R5 is independently (C₁-C₆)-alkyl, aryl, (C₁-C₃)-alkyl-aryl, wherein each alkyl or aryl group is optionally substituted at available positions by (C₁-C₃)-alkyl, CF₃, OH, CN;

R6 is independently H, (C₁-C₆)-alkyl, aryl, (C₁-C₃)-alkyl-aryl, wherein each alkyl or aryl is optionally substituted at available positions by (C₁-C₃)-alkyl, CF₃, OH, CN; or

NR5R6 is a 5- to 12-membered monocyclic ring containing up to 3 heteroatoms selected from O, S and N; or a 6- to 12-membered bicyclic ring system containing up to 4 heteroatoms selected from O, S and N; or a 10- to 18-membered tricyclic ring system containing up to 5 heteroatoms selected from O, S and N; each of these cyclic ring systems optionally substituted at available positions by (c₁-C₆)-alkyl, aryl, heteroaryl, OH, CF₃, O—(C₁-C₆)alkyl.

The invention is also directed to a pharmaceutical composition for treating a disorder or condition selected from the list consisting of acute myocardial infarction; memory processes disorders; dementia; cognition disorders such as Alzheimer's disease; anxiety; attention deficit disorder (ADD); attention-deficit hyperactivity disorder (ADHD); cancers (including cutaneous carcinoma, medullary thyroid carcinoma and melanoma); depression; epilepsy; manic-depressive disorder; Meniere's disease; gastrointestinal disorders; inflammation; migraine; motion sickness; obesity; pain; Parkinson's disease; schizophrenia; septic shock; sleep disorders; respiratory disorders (including allergic rhinitis, nasal congestion and allergic congestion) in a mammal, including a human, that may be treated by administering to said mammal in need of such treatment a compound of formula I as described above, or a pharmaceutically acceptable salt thereof, in a pharmacologically effective dose that is effective in treating such disorder or condition, and a pharmaceutically acceptable carrier.

The invention is also directed to a method of treatment of a disorder or condition selected from the list consisting of acute myocardial infarction; memory processes disorders; dementia; cognition disorders such as Alzheimer's disease; anxiety; attention deficit disorder (ADD); attention-deficit hyperactivity disorder (ADHD); cancers (including cutaneous carcinoma, medullary thyroid carcinoma and melanoma); depression; epilepsy; manic-depressive disorder; Meniere's disease; gastrointestinal disorders; inflammation; migraine; motion sickness; obesity; pain; Parkinson's disease; schizophrenia; septic shock; sleep disorders; respiratory disorders (including allergic rhinitis, nasal congestion and allergic congestion) in a mammal, including a human, that may be treated by administering to said mammal in need of such treatment a compound of formula I as described above, or a pharmaceutically acceptable salt thereof, that is effective in treating such disorder or condition, and a pharmaceutically acceptable carrier.

Preferred embodiments of the present invention include the compounds of formula (I) in which:
(A) R1 and R2 are independently methyl;
   X₂ is 4-fluoro; and
   n is one.
(B) R1 and R2 are independently methyl;
   R3 is hydrogen; and
   n is one.

The most preferred embodiment of the present invention includes the compounds of formula I in which
X₁ is a group of the formula (IV):

—(CH₂)ₘ—NR5R6 (IV);
R1 and R2 are each methyl;
R3 is hydrogen;
X₂ is 4-fluoro;
m is one; and
n is one.

The most preferred compounds of the invention include:

3-[5-(piperidin-1-ylmethyl)-1-(4-fluorophenyl)-1,3-dihydro-2-benzofuran-1-yl]-N,N-dimethylpropan-1-amine, and

3-[4-(4-methylpiperazin-1-ylmethyl)-1-(4-fluorophenyl)-1,3-dihydro-2-benzofuran-1-yl]-N,N-dimethylpropan-1-amine.

Other preferred compounds of the general formula (I) include the following:

3-[5(8-methyl-3,8-diazabicyclo[3.2.1]octan-1-ylmethyl)-1-(4-fluorophenyl)-1,3-dihydro-2-benzofuran-1-yl]-N,N-dimethylpropan-1-amine,

3-[5-(3-methyl-3,8-diazabicyclo[3.2.1]octan-1-ylmethyl)-1-(4-fluorophenyl)-1,3-dihydro-2-benzofuran-1-yl]-N,N-dimethylpropan-1-amine,

3-[5-(2-methyl-2,5-diazabicyclo[2.2.2]octan-1-ylmethyl)-1-(4-fluorophenyl)-1,3-dihydro-2-benzofuran-1-yl]-N,N-dimethylpropan-1-amine,

3-[5-(1,2,6-trimethyl-piperazin-4-ylmethyl)-1-(4-fluorophenyl)-1,3-dihydro-2-benzofuran-1-yl]-N,N-dimethylpropan-1-amine,

3-[5-(3,5-dimethylpiperazin-4-ylmethyl)-1-(4-fluorophenyl)-1,3-dihydro-2-benzofuran-1-yl]-N,N-dimethylpropan-1-amine,

3-[5-(3,3,5,5-tetramethylpiperazin-1-ylmethyl)-1-(4-fluorophenyl)-1,3-dihydro-2-benzofuran-1-yl]-N,N-dimethylpropan-1-amine,

3-[4-(1,2,2,6,6-pentamethylpiperazin-1-ylmethyl)-1-(4-fluorophenyl)-1,3-dihydro-2-benzofuran-1-yl]-N,N-dimethylpropan-1-amine,

3-[5-(4-methyl-octahydro-1H-cyclopenta[b]pyrazin-1-ylmethyl)-1-(4-fluorophenyl)-1,3-dihydro-2-benzofuran-1-yl]-N,N-dimethylpropan-1-amine,

3-[5-(4-methyl-decahydroquinoxalin-1-ylmethyl)-1-(4-fluorophenyl)-1,3-dihydro-2-benzofuran-1-yl]-N,N-dimethylpropan-1-amine,

3-[5-(4-methyl-1,4-diazepin-1-ylmethyl)-1-(4-fluorophenyl)-1,3-dihydro-2-benzofuran-1-yl]-N,N-dimethylpropan-1-amine,

3-[5-(5,7,7-trimethyl-2,5-diazabicylo[2.2.1]heptan-1-ylmethyl)-1-(4-fluorophenyl)-1,3-dihydro-2-benzofuran-1-yl]-N,N-dimethylpropan-1-amine,

3-[5-((4-propan-2-yl)piperazin-1-ylmethyl)-1-(4-fluorophenyl)-1,3-dihydro-2-benzofuran-1-yl]-N,N-dimethylpropan-1-amine,

3-[5-(4-(4-chlorobenzyppiperazin-1-ylmethyl)-1-(4-fluorophenyl)-1,3-dihydro-2-benzofuran-1-yl]-N,N-dimethylpropan-1-amine,

3-[5-(4-(3-chloro-4-fluorobenzyl)piperazin-1-ylmethyl)-1-(4-fluorophenyl)-1,3-dihydro-2-benzofuran-1-yl]-N,N-dimethylpropan-1-amine,

3-[5-(N,N-diethylaminomethyl)-1-(4-fluorophenyl)-1,3-dihydro-2-benzofuran-1-yl]-N,N-dimethylpropan-1-amine,

3-[5-((N,2-dimethyl-propylamino)methyl)-1-(4-fluorophenyl)-1,3-dihydro-2-benzofuran-1-yl]-N,N-dimethylpropan-1-amine,

3-[5-(N,N-dicyclopropylaminomethyl)-1-(4-fluorophenyl)-1,3-dihydro-2-benzofuran-1-yl]-N,N-dimethylpropan-1-amine,

3-[5-(3-(N,N-dimethylamino)pyrrolidin-1-ylmethyl)-1-(4-fluorophenyl)-1,3-dihydro-2-benzofuran-1-yl]-N,N-dimethylpropan-1-amine,

3-[5-(4-(N,N-dimethylamino)piperidin-1-ylmethyl)-1-(4-fluorophenyl)-1,3-dihydro-2-benzofuran-1-yl]-N,N-dimethylpropan-1-amine,

3-[5-(3-dimethylamino-octahydro-1H-indol-ylmethyl)-1-(4-fluorophenyl)-1,3-dihydro-2-benzofuran-1-yl]-N,N-dimethylpropan-1-amine,

3-[5-(octahydro-1H-pyrrolo[3,2-b]pyridin-1-ylmethyl)-1-(4-fluorophenyl)-1,3-dihydro-2-benzofuran-1-yl]-N,N-dimethylpropan-1-amine,

3-[5-(6-methyl-octahydro-1H-pyrrolo[2,3-c]pyridin-1-ylmethyl)-1-(4-fluorophenyl)]-1,3-dihydro-2-benzofuran-1-yl]-N,N-dimethylpropan-1-amine,

3-[5-(octahydro-1H-isoindo1-2-ylmethyl)-1-(4-fluorophenyl)-1,3-dihydro-2-benzofuran-1-yl]-N,N-dimethylpropan-1-amine,

3-[5-(octahydro-cyclopenta[c]pyrrol-2-ylmethyl)-1-(4-fluorophenyl)-1,3-dihydro-2-benzofuran-1-yl]-N,N-dimethylpropan-1-amine,

3-[5-(5-methyl-octahydro-pyrrolo[3,4-c]pyrrol-2-ylmethyl)-1-(4-fluorophenyl)-1,3-dihydro-2-benzofuran-1-yl]-N,N-dimethylpropan-1-amine,

3-[5-(4-methyl-octahydro-pyrrolo[3,4-b]pyrrol-1-ylmethyl)-1-(4-fluorophenyl)-1,3-dihydro-2-benzofuran-1-yl]-N,N-dimethylpropan-1-amine,

3-[5-(hexahydro-1H-furo[3,4-c]pyrrol-5-ylmethyl)-1-(4-fluorophenyl)-1,3-dihydro-2-benzofuran-1-yl]-N,N-dimethylpropan-1-amine,

3-[5-(2,3-dihydro-1H-isoindo1-2-ylmethyl)-1-(4-fluorophenyl)-1,3-dihydro-2-benzofuran-1-yl]-N,N-dimethylpropan-1-amine,

3-[5-(octahydro-pyrano[3,4-c]pyrrol-2-ylmethyl)-1-(4-fluorophenyl)-1,3-dihydro-2-benzofuran-1-yl]-N,N-dimethylpropan-1-amine,

3-[5-(4-azatricyclo[5.2.2.0^{2,6}]undecan-4-ylmethyl)-1-(4-fluorophenyl)-1,3-dihydro-2-benzofuran-1-yl]-N,N-dimethylpropan-1-amine,

3-[5-(azabicyclo[2.2.2]octan-3-ylaminomethyl)-1-(4-fluorophenyl)-1,3-dihydro-2-benzofuran-1-yl]-N,N-dimethylpropan-1-amine,

3-[5-(2-methyl-azabicyclo[2.2.2]octan-3-ylaminomethyl)-1-(4-fluorophenyl)-1,3-dihydro-2-benzofuran-1-yl]-N,N-dimethylpropan-1-amine,

3-[5-(N-methyl-azabicyclo[2.2.2]octan-3-ylaminomethyl)-1-(4-fluorophenyl)-1,3-dihydro-2-benzofuran-1-yl]-N,N-dimethylpropan-1-amine,

3-[5-(4-(N,N-dimethylamino)piperidin-1-ylmethyl)-1-(4-fluorophenyl)-1,3-dihydro-2-benzofuran-1-yl]-N,N-dimethylpropan-1-amine,

3-[5-(1,4′-bipiperidin-1-ylmethyl)-1-(4-fluorophenyl)-1,3-dihydro-2-benzofuran-1-yl]-N,N-dimethylpropan-1-amine,

3-[5-(2,6-dimethylmorpholin-4-ylmethyl)-1-(4-fluorophenyl)-1,3-dihydro-2-benzofuran-1-yl]-N,N-dimethylpropan-1-amine,

3-[5-((N-cyclohexylmethylamino)methyl)-1-(4-fluorophenyl)-1,3-dihydro-2-benzofuran-1-yl]-N,N-dimethylpropan-1-amine,

3-[5-(4-(1,2-benzisothiazol-3-yl)piperazin-1-ylmethyl)-1-(4-fluorophenyl)-1,3-dihydro-2-benzofuran-1-yl]-N,N-dimethylpropan-1-amine,

3-[5-(4-(1,2-thiazol-3-yl)piperazin-1-ylmethyl)-1-(4-fluorophenyl)-1,3-dihydro-2-benzofuran-1-yl]-N,N-dimethylpropan-1-amine,

3-[5-(4-(1H-1,2,4-triazol-3-yl)piperazin-1-ylmethyl)-1-(4-fluorophenyl)-1,3-dihydro-2-benzofuran-1-yl]-N,N-dimethylpropan-1-amine,

3-[5-(4-(1,3-oxazol-2-yl)piperazin-1-ylmethyl)-1-(4-fluorophenyl)-1,3-dihydro-2-benzofuran-1-yl]-N,N-dimethylpropan-1-amine,

3-[5-(4-(1H-imidazol-2-yl)piperazin-1-ylmethyl)-1-(4-fluorophenyl)-1,3-dihydro-2-benzofuran-1-yl]-N,N-dimethylpropan-1-amine,

3-[5-(4-(1,3-thiazol-2-yl)piperazin-1-ylmethyl)-1-(4-fluorophenyl)-1,3-dihydro-2-benzofuran-1-yl]-N,N-dimethylpropan-1-amine,

3-[5-(4-(pyrimidin-2-yl)piperazin-1-ylmethyl)-1-(4-fluorophenyl)-1,3-dihydro-2-benzofuran-1-yl]-N,N-dimethylpropan-1-amine,

3-[5-(4-(pyrimidin-4-yl)piperazin-1-ylmethyl)-1-(4-fluorophenyl)-1,3-dihydro-2-benzofuran-1-yl]-N,N-dimethylpropan-1-amine, and

3-[5-(4-(pyrimidin-5-yl)piperazin-1-ylmethyl)-1-(4-fluorophenyl)-1,3-dihydro-2-benzofuran-1-yl]-N,N-dimethylpropan-1-amine.

A preferred use for compounds of formula (I) is in the treatment of a disorder or condition selected from the list consisting of acute myocardial infarction; memory processes disorders; dementia; cognition disorders such as Alzheimer's disease; anxiety; attention deficit disorder (ADD); attention-deficit hyperactivity disorder (ADHD); cancers (including cutaneous carcinoma, medullary thyroid carcinoma and melanoma); depression; epilepsy; manic-depressive disorder; Meniere's disease; gastrointestinal disorders; inflammation; migraine; motion sickness; obesity; pain; Parkinson's disease; schizophrenia; septic shock; sleep disorders; respiratory disorders (including allergic rhinitis, nasal congestion and allergic congestion) in a mammal, including a human. More specifically, the compounds of formula I are useful in the treatment of the human diseases and disorders listed above, as defined in the Diagnostic and Statistical Manual of Mental Disorders, 4^{th} Edition (DSM-IV) published by the American Psychiatric Association, June 2000.

### DETAILED DESCRIPTION OF THE INVENTION

The compounds of formula (I) may be prepared as described in the following reaction schemes and discussions. Unless otherwise indicated, X₁, X₂, R1, R2, R3, R5 and R6, and structural formulae II, Ill, IV, VI, VII in the reaction schemes and discussion that follow are as defined above.

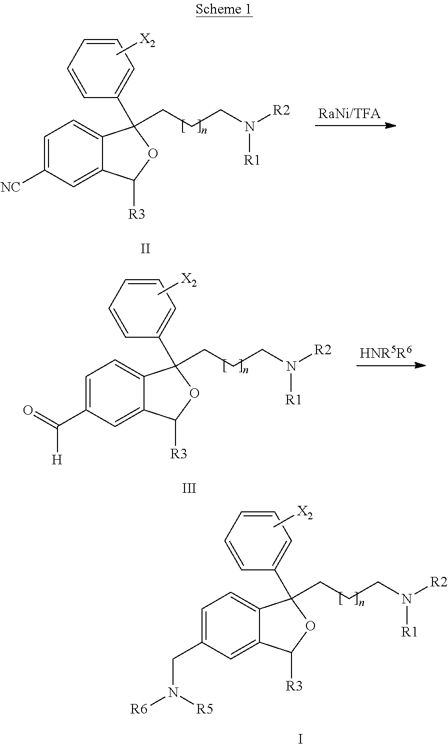

According to Scheme 1, compounds of the general formula (I) may be prepared from the compound of general formula II by conversion of the nitrile (—CN) group to an aldehyde of general formula III, which is then reacted with an amine of the general formula HNR5R6 to produce the title compound of general formula (I). The conversion of the nitrile group of compound II can be achieved using specific and selective conditions described in the chemical literature.

In the first step, the nitrile compound of formula II is converted to the corresponding aldehyde of formula III using, for example, a mixture of Raney nickel in a protic solvent, for example trifluoroacetic acid (TFA), according to the procedure of T. van Es and B. Staskun (Organic Synthesis, 1988, Coll. Vol. 6, 631). The reaction is conducted by stirring the mixture at atmospheric pressure for a period in the range of about 1 hr to about 3 days, preferably 4-18 hr at a temperature in the range of about 20° C. to about 100° C., preferably at the reflux temperature of the solvent, until the reaction is judged to be complete or until the starting nitrile II is no longer detectible, using, e.g., thin layer chromatography (tic). Other reaction conditions to effect the conversion of the compound of formula II to the intermediate aldehyde of formula III include the Stevens reduction, in which the nitrile is first treated with HCl, followed by reduction with anhydrous tin (II) chloride (i.e., SnCl₂) (see Rabinovitz, in The Chemistry of the Cyano Group, J. Wiley and Sons, New York, 1970, 307-340). Other reagents to effect this transformation include those listed in Larock, (Comprehensive Organic Transformations; Wiley-VCH, New York, 1989, 993). Other methods to produce aldehydes from nitriles involve the use of reducing reagents like lithium aluminum hydride (LiAIH₄), LiAIH(OEt)₃ (see Malek, Organic Reactions, 1988, 36: pp 249-59, pp 287-9, pp 438-48), DIBAL (see Marshall, et al, Journal of Organic Chemistry, 1970, 35:858), NaAIH₄, sodium hypophosphate, Raney nickel in aqueous acetic acid-pyridine or formic acid (see Khai, Journal of Organic Chemistry 1989 54:949). Diisobutylaluminum hydride is also a useful reagent that has been used to prepare aldehydes from nitriles (see LeBel, et al, Journal of the American Chemical Society, 1964, 86:3759; Stevens, et al, Journal of Organic Chemistry, 1972, 37:2138); triethoxyaluminum hydride has also been used successfully (see Brown, et al, Journal of the American Chemical Society, 1964, 86:1085)

The intermediate aldehyde of formula III may then be reacted with a primary or secondary amine of the general formula HNR5R6, wherein R5 and R6 are as previously defined, using methods described in the literature and known to one skilled in the art of organic synthesis, to prepare the title compounds of the present invention. One method to prepare compounds of general formula (I) from intermediate aldehydes of general formula III involves reaction of the aldehyde and an amine of general formula HNR5R6 in the presence of titanium tetrachloride (TiCI₄) and triethylamine (TEA) in dichloromethane to form an intermediate imine, followed by reduction of said imine with sodium cyanoborohydride (NaCNBH₄) in methanol. This process, referred to as a Borch reductive amination, is described by Borch (e.g., see Borch RF, et al, Journal of the American Chemical Society, 1969, 91:3996-7; Borch, et al, Journal of Organic Chemistry, 1977, 42:1225-7; Barney C L, et al, Tetrahedron Letters, 1990, 31:5547-50). The use of indium trichloride (InCl₃) followed by reduction using triethylsilane in methanol is described by O.-Y. Lee, et al (Journal of Organic Chemistry, 2008, 73:8829). The imine may also be prepared by combining the aldehyde II and an amine of general formula HNR5R6 in an inert solvent (e.g., toluene) in the presence of TiCl₄ or molecular sieves and stirring or refluxing the mixture to eliminate water formed in the reaction. The intermediate imine so obtained may then be isolated and treated with a reducing agent, e.g., NaBH₄, NaBH₃CN, in a solvent such as methanol to generate the desired final product of formula (I).

The starting materials for this process, i.e., compounds of the general formula II, are available using procedures described in the chemical and patent literature. For example, the compound of formula II, wherein n=1, R1═CH3, R2═CH3, R3═H, X₂ is 4-fluoro and X₁ is a CN group located at the 5-position of the benzofuran ring, is commercially available in racemic form (i.e., a 50:50 mixture of (R)— and (S)— isomers) and sold as the antidepressant citalopram or as a single (S)—isomer (marketed as the antidepressant escitalopram). Procedures for the syntheses of these compounds have also been published in the patent and chemical literature (e.g., see M. Pitts, Tetrahedron, 2006, 62:4705-4708; P. Zhang, et al, J. Medicinal Chem., 2010, 53:6112-6121; N. Periyandi, et al, PCT Int. Appl., 2006, WO-2006021971; T. Ikemoto and Y. Watanabe, PCT Int. Appl., 2005, WO-2005082842; H. Ahmadian and H. Petersen, PCT Int. Appl., 2003, WO-2003051861; H. Petersen, PCT Int. Appl. 2001, WO-2001068631; L. Dall'Asta, et al, PCT Int. Appl., 2000, WO-2000023431).

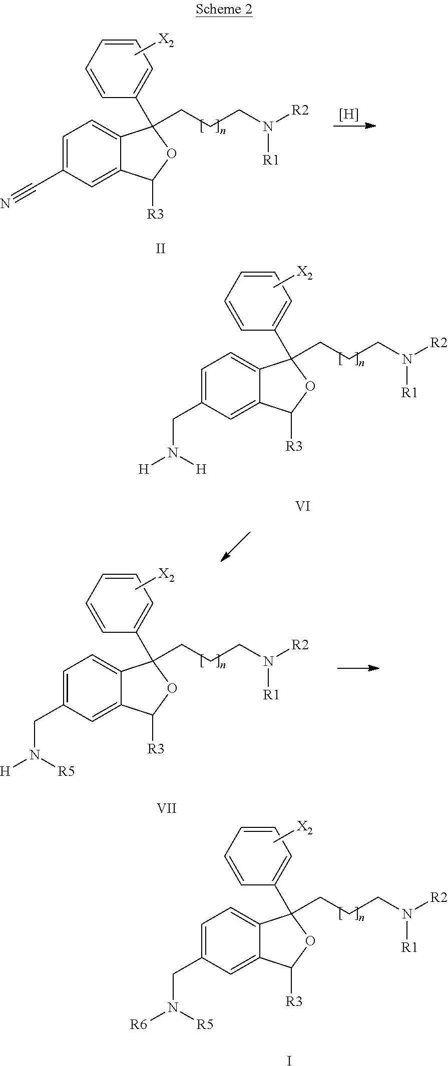

According to Scheme 2 above, a compound of the general formula II can be converted into a compound of general formula VI through a reduction procedure, using reagents and conditions well known to one skilled in the art. Thus, the nitrile of general formula II may be treated with a reducing agent such as lithium aluminum hydride (LiAIH₄) in THF or diethyl ether (e.g., see Evans, et al, Journal of Organic Chemistry, 1974, 39:914), or diisopropylaminoborane (see Haddenham, et al, Journal of Organic Chemistry, 2009, 74:1964-1970), to prepare the primary amine of general formula VI. General references for hydrogenation procedures to convert nitriles to amines can also be found in Rylander, P. N., Catalytic Hydrogenation in Organic Syntheses, Academic Press, New York, N.Y., 1979.

The primary amine compound of general formula VI so obtained may then be reacted in a variety of ways to generate the intermediate of general formula VII, e.g., through the alkylation of the primary amine using a compound R5-L, wherein L is a leaving group, e.g., Cl, Br, I, OSO₂Me, OSO₂CF₃, etc. This reaction is often performed with an excess of the amine or in the presence of a tertiary amine such as triethylamine to perform as an acid scavenger.

Alternatively, the intermediate of formula VI may be reacted under reductive alkylation conditions with an aldehyde or ketone to create an intermediate imine, followed by reduction of the C═N bond to produce the desired secondary amine compound of general formula VII, wherein R5 is as previously defined above for the conversion of Ill to I (Scheme 1). Compounds VII so obtained may be further reacted in the same manner as above with a second reagent (i.e., R6-L) to produce the corresponding tertiary amine of general formula (I) wherein R5 and R6 are as previously defined.

Where cis- and trans- isomers are possible for an embodiment of the inventive compounds of formula I, both cis- and trans- isomers (i.e., diastereomers) are within the scope of this invention. Similarly, when R— and S—, or (+)- and (−)-, or d- and I- isomers (i.e., enantiomers) are possible for an embodiment of the inventive compounds of formula (I), each and every one of said isomers are within the scope of this invention.

The term “alkyl” refers to straight or branched chains of carbon atoms. Exemplary alkyl groups are C₃-C₁₀ alkyl groups which include methyl, ethyl, propyl, isopropyl, butyl, isobutyl, pentyl, isopentyl, hexyl and the like, including all regio-isomeric forms thereof, and straight and branched chain forms thereof. The term “alkyl” is also used to denote straight or branched chains of carbon atoms having one or more carbon-carbon double bonds, such as vinyl, allyl, butenyl and the like, as well as straight and branched chains of carbon atoms having one or more carbon-carbon triple bonds, such as ethynyl, propargyl, butynyl, and the like.

The term “aryl” denotes a cyclic, aromatic hydrocarbon. Examples include phenyl, naphthyl, anthracenyl, phenanthracenyl, and the like.

The terms “alkoxy” and “aryloxy” denote “O-alkyl” and “O-aryl”, respectively. The term “cycloalkyl” denotes a cyclic group of carbon atoms, where the ring formed by the carbon atoms may be saturated or may comprise one or more carbon double bonds in the ring. Examples of cycloalkyl groups include cyclopropyl, cyclobutyl, cyclopentyl, cyclohexyl, cycloheptyl, and the like as well as cyclopentenyl, cyclopentadienyl, cyclohexenyl, cyclohexadienyl, and the like. As used herein, the term “cycloalkyl” is also intended to denote a cyclic group comprising at least two fused rings, such as adamantyl, decahydronaphthalinyl, norbornanyl, where the cyclic group may also have one or more carbon-carbon double bonds in one or more rings, such as in bicyclo(4.3.0)nona-3,6(1)-dienyl, dicyclopentadienyl, 1,2,3,4-tetrahydronaphthalinyl (tetralinyl), indenyl, and the like.

The term “one or more substituents” as used herein, refers to a number of substituents that equals from one to the maximum number of substituents possible based on the number of available bonding sites.

The terms “halo” and “halogen”, as used herein, unless otherwise indicated, include chloro, fluoro, bromo and iodo.

The term “heteroaryl” denotes a monocyclic or bicyclic aromatic group wherein one or more carbon atoms are replaced with heteroatoms selected from the group consisting of nitrogen, oxygen, and sulfur. If the heteroaryl group contains more than one heteroatom, the heteroatoms may be the same or different. Preferred heteroaryl groups are five- to fourteen-member rings that contain from one to four heteroatoms independently selected from oxygen, nitrogen, and sulfur. Examples of preferred heteroaryl groups include benzo[b]thienyl, chromenyl, furyl, imidazolyl, indazolyl, indolizinyl, indolyl, isobenzofuranyl, isoindolyl, isoquinolinyl, isothiazolyl, isoxazolyl, napthylidinyl, oxadiazolyl, oxazinyl, oxazolyl, phthalazinyl, pteridinyl, purinyl, pyranyl, pyrazinyl, pyrazolyl, pyridazinyl, pyridinyl, thiazolyl, isothiazolyl, benzothiazolyl, benzisothiazoly, thiadiazolyl, triazolyl, triazinyl and tetrazolyl.

The term “treating”, as used herein, refers to reversing, alleviating, inhibiting the progress of, or preventing the disorder or condition to which such term applies, or preventing one or more symptoms of such disorder or condition. The term “treatment”, as used herein, refers to the act of treating, as “treating” is defined immediately above.

The compounds of formula (I) of the present invention may also contain functional groups or heterocyclic ring systems that may exist in one or more tautomeric forms. The present invention includes within its scope all such tautomeric forms, including mixtures of such forms.

The compounds of the present invention may have optical centers and therefore may occur in different enantiomeric configurations. Formula (I), as depicted above, includes all enantiomers, diastereomers, and other stereoisomers of the compounds depicted in structural formula (I), as well as racemic and other mixtures thereof. Individual isomers can be obtained by known methods, such as optical resolution, optically selective reaction, or chromatographic separation in the preparation of the final product or any of its intermediates.

The compounds of formula (I) may also exist in the form of cis- or trans- isomers with respect to configuration on the furan ring of formula (I). Such cis- and trans- isomers are also considered to be within the scope of the present invention,

The present invention also includes isotopically labeled compounds, which are identical to those recited in formula (I), but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, sulfur, phosphorus, fluorine, and chlorine, such as ²H, ³H, ¹¹C, ¹³ C, ¹⁴C, ¹³N, ¹⁵N, ¹⁷O, ¹⁸O, ³⁵S, ³¹P, ³²P, ³¹P, ¹⁸F, and ³⁷Cl, respectively. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with isotopes such as deuterium, i.e., ²H, can afford certain therapeutic advantages resulting from greater metabolic stability, for example increased in vivo half-life or reduced dosage requirements and hence may be preferred in some circumstances. Isotopically labeled compounds of formula (I) of this invention and prodrugs thereof can generally be prepared by carrying out the procedures disclosed in the Schemes and/or the examples and Preparations below, by substituting a readily available isotopically labeled reagent for a non-isotopically labeled reagent.

Compounds of the present invention, prodrugs thereof, and pharmaceutically acceptable salts of said compounds, or of said prodrugs which contain the aforementioned isotopes and/or other isotopes of other atoms are within the scope of this invention.

A “unit dosage form” as used herein is any form that contains a unit dose of the compound of formula (I). A unit dosage form may be, for example, in the form of a tablet or a capsule. The unit dosage form may also be in liquid form, such as a solution or suspension.

The compositions of the present invention may be formulated in a conventional manner using one or more pharmaceutically acceptable carriers. Thus, the active compounds of the present invention may be formulated for oral, buccal, intranasal, parenteral (e.g., intravenous, intramuscular or subcutaneous) or rectal administration or in a form suitable for administration by inhalation or insufflations.

For oral administration, the pharmaceutical compositions may take the form of, for example, tablets or capsules prepared by conventional means with pharmaceutically acceptable excipients such as binding agents (e.g., pre-gelatinized maize starch, polyvinylpyrrolidone or hydroxypropyl methylcellulose), fillers (e.g., lactose, microcrystalline cellulose or calcium phosphate); lubricants (e.g., magnesium stearate, talc, or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The active compounds (I) of the invention may be formulated for parenteral administration by injection, including using conventional catheterization techniques or infusion.

Formulations for injection may be presented in unit dosage form, e.g., in ampoules or in multi-dose containers, with an added preservative. The compositions may take such forms as suspensions, solutions or emulsions in oily or aqueous vehicles, and may contain formulating agents such as suspending, stabilizing and/or dispensing agents. Alternatively, the active ingredient may be in powder form for reconstitution with a suitable vehicle, e.g., sterile pyrogen-free water, before use.

The active compounds of the invention (I) may also be formulated in rectal compositions such as suppositories or retention enemas, e.g., containing conventional suppository bases such as cocoa butter or other glycerides.

For intranasal administration by inhalation, the active compounds of the invention (I) are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane (CCl₂F₂), trichlorofluoromethane (CCl₃F), difluorotetrachloroethane (ClF₂CCl₃), carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insulator may be formulated containing a powder mix of a compound of the invention and a suitable powder base such as lactose or starch.

A proposed dose of the active compounds (I) of the invention for oral, parenteral or buccal administration to the average adult human for the treatment of the conditions referred to above (e.g., depression) is from about 0.1 mg/kg to about 100 mg/kg of the active ingredient per unit dose which could be administered, for example, one to four times per day. Toxicity concerns at the higher level may restrict intravenous (i.v.) dosages to a lower level, such as up to about 10 mg/kg. A dose of about 0.1 mg/kg to about 100 mg/kg may be employed for oral (p.o.) administration. Typically, a dosage from about 0.1 mg/kg to about 10 mg/kg may be employed for intramuscular (i.m.) injection. Preferred dosages are in the 1.0 mg/kg to about 100 mg/kg range, and more preferably in the 5 mg/kg to about 50 mg/kg range for i.v. or p.o. administration. The duration of the treatment is usually once per day for a period of three days to three weeks, or until the condition is essentially controlled. Lower doses given less frequently can be used prophylactically to prevent or reduce the incidence of recurrence of the infection.

Aerosol formulations for treatment of the conditions referred to above (e.g., depression) in the average human are preferably arranged such that each metered dose or “puff” of aerosol contains 0.1 micrograms to 100 micrograms of the compound of the invention. The overall daily dose with an aerosol will be within the range of 0.1 mg/kg to about 100 mg/kg, and preferably in the range of 1.0 mg/kg to about 25 mg/kg. Administration may be several times daily, for example 2, 3, 4 or 8 times, giving for example 1, 2 or 3 doses each time.

As an example, the mammal in need of treatment or prevention may be a human. As another example, the mammal in need of treatment or prevention may be a mammal other than a human.

In so far as the compounds of formula (I) of this invention are basic compounds, they are capable of forming a variety of different salts with various inorganic and organic acids. Although such salts must be pharmaceutically acceptable for administration to animals, including humans, it is often desirable in practice to initially isolate the base compound from the reaction mixture as a pharmaceutically unacceptable salt, then isolate the base by treatment of the salt with an alkaline reagent and finally convert the isolated free base compound to a pharmaceutically acceptable acid addition salt.

The acids which are used to prepare the pharmaceutically acceptable acid salts of the active compound used in formulating the pharmaceutical composition of this invention that are basic in nature are those which form non-toxic acid addition salts, e.g., salts containing pharmacologically acceptable anions. Non-limiting examples of the salts include the acetate, benzoate, beta-hydroxybutyrate, bisulfate, bisulfite, bromide, butyne-1,4-dioate, caproate, chloride, chlorobenzoate, citrate, dihydrogen phosphate, dinitrobenzoate, fumarate, glycollate, heptanoate, hexyne-1,6-dioate, hydroxybenzoate, iodide, lactate, maleate, malonate, mandelate, metaphosphate, methanesulfonate, methoxybenzoate, monohydrogen phosphate, naphthalene-1-sulfonate, naphthalene-2-sulfonate, oxalate, phenyl butyrate, phenylpropionate, phosphate, phthalate, phenylacetate, propanesulfonate, propiolate, propionate, pyrophosphate, pyrosulfate, sebacate, suberate, succinate, sulfate, sulfite, sulfonate, tartrate, xylenesulfonate, trifluoroacetate, acid phosphate, acid citrate, bitartrate, succinate, gluconate, saccharate, nitrate, methanesulfonate, and pamoate [i.e., 1,1′-methylene-bis-(2-hydroxy-3-naphthoate)] salts.

Also included within the scope of this invention are solvates and hydrates of compounds of formula I and their pharmaceutically acceptable salts. The present invention includes within its scope all possible stoichiometric and non-stoichiometric forms.

In the examples that follow, the abbreviations used are intended to have the following, general meaning:

bm: broad multiplet (NMR)

bs: broad singlet (NMR)

d: doublet (NMR)

dd: doublet of doublets (NMR)

d.e.: diatomaceous earth, filtering agent

calcd.: calculated value

equiv: equivalent

J: coupling constant (NMR)

HPLC: high pressure liquid chromatography

m: multiplet (NMR)

min: minute(s)

m/z: mass to charge ratio (mass spectroscopy)

obsd: observed value

Rf: retention factor (chromatography)

RT: retention time (chromatography)

rt: room temperature (typically 25 ° C.)

s: singlet (NMR)

t: triplet (NMR),

T: temperature

tic: thin layer chromatography

TFA: trifluoroacetic acid

THF: tetrahydrofuran

Solvents were purchased and used without purification. Yields were calculated for material judged homogeneous by thin layer chromatography and NMR. Thin layer chromatography was performed on Kieselgel plates eluting with the indicated solvents, visualized by using a 254 nm UV lamp, or stained with an aqueous KMnO₄ solution, an ethanolic solution of 12-molybdophosphoric acid, or an iodoplatinate spray reagent (available from Sigma-Aldrich Co., St. Louis, Mo.).

Nuclear Magnetic Resonance (NMR) spectra were acquired on 90- or 400-MHz NMR

Spectrometers. Chemical shifts for proton NMR spectra are reported in parts per million (ppm) relative to the singlet of CDCl₃ at 7.24 ppm.

### PREPARATION 1

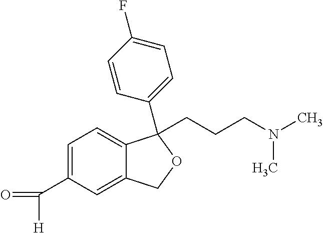 [SMILES: CN(C)CCCC1(c2ccc(F)cc2)OCc2cc(C=O)ccc21]

### 1-[3-(dimethylam ino)propyl]-1-(4-fluorophenyl)-1,3-dihydro-2-benzofuran-5-carbaldehyde

A mixture of citalopram hydrobromide (1.0 g, 2.5 mmol, MW=405.3, TCI America, Portland, Oreg.) and Raney nickel (50% H₂O, Sigma-Aldrich Co., St. Louis, Mo.) in 25 mL formic acid (96%) was refluxed for 20 hr, cooled to room temperature and carefully poured over 300 mL of saturated aqueous sodium carbonate (caution vigorous foaming) and adjusted to a final pH of 10.0. After extraction with EtOAc (25 mL×3) the organic layers were washed with saturated NaCl and dried with MgSO₄. After filtration, the solvent was removed in vacuo to leave a pale yellow oil, 0.70 g (%). NMR (300 MHz, CDCl₃) was consistent with published spectral data.

### EXAMPLE 1

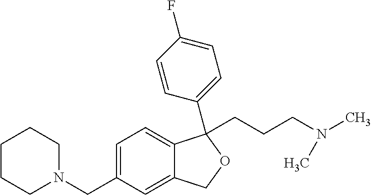 [SMILES: CN(C)CCCC1(c2ccc(F)cc2)OCc2cc(CN3CCCCC3)ccc21]

### 3-[5-(Piperidin-1-yl)methyl)-1-(4-fluorophenyl)-1,3-dihydro-2-benzofuran-1-yl]-N,N-dimethyl-propan-1-amine

A mixture of 1-[3-(dimethylamino)propyl]-1-(4-fluorophenyl)-1,3-dihydro-2-benzofuran-5-carbaldehyde (250 mg, 0.76 mmol) from Procedure 1, anhydrous piperidine (0.075 ml, 0.76 mmol, MW=85.15) and acetic acid (0.05 mL, 0.87 mmol, MW=60.05) in tetrahydrofuran (10 mL) was stirred for 30 min at room temperature and then treated with sodium triacetoxyborohydride (STAB, 403 mg, 1.9 mmol, MW=211.94, Sigma-Aldrich Co., St. Louis, Mo.) and left to stir for 24 h, resulting in a milky white cloudy suspension. A tic (EtOAc:CH₃CN:TEA (10:1:0.5)) showed no starting aldehyde present. The reaction mixture was partitioned between EtOAc and water and the pH adjusted to ˜11.0 with aqueous saturated Na₂CO₃. The organic layer was removed, the aqueous layer re-extracted twice with EtOAc and the combined EtOAc extracts were dried over MgSO₄, filtered and concentrated in vacuo.

The crude material was chromatographed using silica gel (230-400 mesh) and eluting with EtOAc:CH₃CN:TEA (10:1:0.5). Fractions containing the desired product were combined and concentrated in vacuo to a colorless oil, 29 mg.

NMR (CDCl₃, 90 MHz) δ 6 7.44 (m, 2H), 7.19 (m, 3H), 6.97 (m, 2H), 5.12 (bs, 2H), 3.43 (s, 2H), 2.36-2.05 (m, 8H), 2.13 (s, 6H), 1.49-1.26 (m, 8H).

### EXAMPLE 2

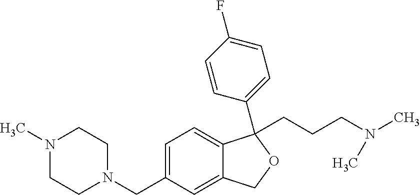

### 3-[5-((4-methylpiperazin-1-yl)methyl)-1-(4-fluorophenyl)-1,3-dihydro-2-benzofuran-1-yl]-N,N-dimethylpropan-1-amine.

Starting with 1-[3-(dimethylamino)propyl]-1-(4-fluorophenyl)-1,3-dihydro-2-benzofuran-5-carbaldehyde and N-methylpiperazine (Sigma-Aldrich Co., St. Louis, Mo), the title compound was prepared in the same manner as for Example 1, producing a colorless oil, 17 mg.

NMR (CDCl₃, 90 MHz) δ 7.44 (m, 2H), 7.20 (m, 3H), 6.97 (m, 2H), 5.12 (bs, 2H), 3.48 (s, 2H), 2.45 (s, 6H), 2.27-2.05 (m, 9H), 2.13 (s, 6H), 1.42 (m, 2H).

### Histamine Receptor Affinity

The compounds from the above Examples were tested for activity vs. histamine receptor subtypes (i.e., H1, H2, H3), at an initial concentration of 10 μM. Binding activity measurement was performed by the NIMH Psychoactive Drug Screening Program (PDSP) at the University of North Carolina Chapel Hill, N.C. The procedures employed by the PDSP are described in the NIMH PDSP Assay Protocol Book Version II (see, http://pdsp.med.unc.edu/PDSP %20Protocols %2011%202013-03-28.pdf). Data (e.g., H3antagonist activity) may also be generated using a commercially available kit from Invitrogen/Thermo Fisher Scientific Inc. (Tango™ H3-bla U2OS DA Assay Kit). Data for serotonin reuptake activity (SERT, % inhibition at 10 μM drug concentration; calculated Ki) were also generated by PDSP.

DATA

| | | Inhibition | Ki |
|---|---|---|---|
| Example | Receptor Subtype | (% at 10 μM) | (nM) |
| 1 | H1 | 89.9 | 101 |
| 1 | H3 | 99.6 | 20 |
| 1 | SERT | 98.8 | 29 |
| 2 | H1 | 89.8 | 144 |
| 2 | H3 | 89.8 | 155 |
| 2 | SERT | 99.3 | 31 |

We claim:

1. A method of treatment of a disorder or condition selected from the list consisting of acute myocardial infarction; anxiety; dementia; cognition disorders such as Alzheimer's disease; attention deficit disorder (ADD); attention-deficit hyperactivity disorder (ADHD); cancers (including cutaneous carcinoma, medullary thyroid carcinoma and melanoma); memory processes disorders; depression; manic-depressive disorder; epilepsy; Meniere's disease; gastrointestinal disorders; inflammation; migraine; motion sickness; obesity; pain; Parkinson's disease; schizophrenia; sleep disorders; septic shock; respiratory disorders (including allergic rhinitis, nasal congestion and allergic congestion) in a mammal, including a human, the method comprising administering to said mammal in need of such treatment a therapeutically effective amount of a compound of the formula (I): 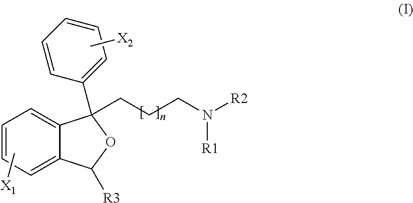
or a pharmaceutically acceptable salt thereof, wherein:
X₁ is a group of the formula:
—(CH₂)ₘ—NR5R6
X₂ is H, Cl, or F;
R1 and R2 are independently hydrogen or methyl;
R3 is hydrogen or methyl;
m is one;
n is zero, one or two;
R5 is independently (C₁-C₆)-alkyl, aryl, (C₁-C₃)-alkyl-aryl, each alkyl or aryl being optionally substituted at available positions by alkyl, CF₃, OH, CN and halogen;
R6 is independently H, (C₁-C₆)-alkyl, aryl, (C₁-C₃)-alkyl-aryl, each alkyl or aryl being optionally substituted at available positions by alkyl, CF₃, OH, CN and halogen; or
NR5R6 is a 5- to 12-membered monocyclic ring containing up to 3 heteroatoms selected from O, S and N; or a 6- to 12-membered bicyclic ring system containing up to 4 heteroatoms selected from O, S and N; or a 10- to 18-membered tricyclic ring system containing up to 5 heteroatoms selected from O, S and N; each of these cyclic ring systems optionally substituted at available positions by (C₁-C₆)-alkyl, aryl, heteroaryl, OH, CF₃, and O—(C₁-C₆)alkyl.

2. The pharmaceutical composition for treating a disorder or condition selected from the list consisting of acute myocardial infarction; anxiety; dementia; cognition disorders such as Alzheimer's disease; attention deficit disorder (ADD); attention-deficit hyperactivity disorder (ADHD); cancers (including cutaneous carcinoma, medullary thyroid carcinoma and melanoma); memory processes disorders; depression; manic-depressive disorder; epilepsy; Meniere's disease; gastrointestinal disorders; inflammation; migraine; motion sickness; obesity; pain; Parkinson's disease; schizophrenia; sleep disorders; septic shock; respiratory disorders (including allergic rhinitis, nasal congestion and allergic congestion) in a mammal, including a human, comprising a compound of formula I as described in claim 1, or a pharmaceutically acceptable salt thereof, and a pharmaceutically acceptable carrier.

3. (canceled)

4. (canceled)

5. The method of claim 1 wherein R5 is independently (C₁-C₆)-alkyl, aryl, (C₁-C₃)-alkyl-aryl, each alkyl or aryl optionally substituted at available positions by alkyl, CF3, OH, CN and halogen.

6. The method of claim 1 wherein R6 is independently (C₁-C₆)-alkyl, aryl, (C₁-C₃)-alkyl-aryl, each alkyl or aryl optionally substituted at available positions by alkyl, CF3, OH, CN and halogen.

7. The method of claim 1 wherein NR5R6 is a 5- to 12-membered monocyclic ring containing up to 3 heteroatoms selected from O, S and N; or a 6- to 12-membered bicyclic ring containing up to 3 heteroatoms selected from O, S and N; or a 10- to 18-membered tricyclic ring containing up to 3 heteroatoms selected from O, S and N.

8. The method of claim 1 wherein the mammal is a human.
